(19) United States
(12) Patent Application Publication
(10) Pub. No.: US 2026/0083512 A1
(43) Pub. Date: Mar. 26, 2026

(54) HANDOFF OF ELECTRONIC ACCESSORY BETWEEN SURGICAL CONSOLES

(71) Applicant: Alcon Inc., Fribourg (CH)

(72) Inventor: Dan Teodorescu, Irvine, CA (US)

(21) Appl. No.: 19/326,183

(22) Filed: Sep. 11, 2025

Related U.S. Application Data
(60) Provisional application No. 63/698,141, filed on Sep. 24, 2024.

(51) Int. Cl. A61B 34/20

(52) U.S. Cl. CPC A61B 34/20; A61B 2034/2074

(57) ABSTRACT

In certain embodiments, a surgical system includes an input device, a first surgical console, and a second surgical console. Each surgical console includes a respective controller that controls operations of the surgical console. During a first time period, the first surgical console is assigned ownership of the input device and is controlled based on input(s) therefrom. The second surgical console is non-responsive to the input(s) during the first time period. The first surgical console transfers ownership to the second surgical console responsive to receiving a transfer indication. During a second time period, the second surgical console is assigned ownership of the input device and is controlled based on input(s) therefrom. The first surgical console is non-responsive to the input(s) during the second time period.

### INTRODUCTION

In modern surgical procedures, the use of multiple surgical consoles has become increasingly prevalent. Each surgical console is typically operated by a surgeon or technician to perform functions specific to different surgical procedures. One advantage of employing multiple surgical consoles is the ability to conduct complex surgeries with greater efficiency and accuracy. Providing the functionality through the distinct surgical consoles allows surgeons to perform multiple intricate procedures with enhanced precision and control.

Moreover, in some cases, the use of multiple surgical consoles may facilitate collaborative surgery, where multiple surgeons can work simultaneously on different aspects of the surgical procedure. This collaboration can enhance surgical outcomes by leveraging the expertise of multiple specialists and reducing overall operative time.

Certain functions of a surgical console may be controlled using electronic accessories, such as foot controllers (e.g., footswitches), remote controls, and so forth. However, using numerous electronic accessories in an operating or clinical environment can present several challenges, such as being functionally redundant (e.g., multiple foot controllers used for different surgical consoles), being an inefficient use of the limited amount of space of the environment, the possibility of interference between the electronic accessories, and increased complexity of communications with the electronic accessories.

### BRIEF SUMMARY

The present disclosure relates generally to techniques for handing off an electronic accessory between surgical consoles. In some embodiments, a surgical system includes an input device (e.g., an electronic accessory such as a foot controller or remote control), and a first surgical console and a second surgical console that are communicatively coupled with each other.

The first surgical console includes a first controller that controls operation of the first surgical console based on input(s) received from the input device during a first time period. During the first time period, the first surgical console is assigned ownership of the input device (stated another way, the first controller claims ownership or control of the input device), and the second surgical console is non-responsive to the first input. The first controller is further configured to transfer the ownership of the input device to the second surgical console responsive to receiving or generating a transfer indication. The transfer indication may be received from a user interface (e.g., of the first surgical console, the second surgical console, or another electronic device). In some cases, receiving or generating the transfer indication may be responsive to the completion of a phase of the surgical procedure.

The second surgical console comprises a second controller that controls operations of the second surgical console based on a second input received from the input device during a second time period. During the second time period, the second surgical console is assigned ownership of the input device (stated another way, the second controller claims ownership or control of the input device) and the first surgical console is non-responsive to the second input.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates an exemplary surgical console of a surgical system, according to certain embodiments.

FIG. 2 is a block diagram illustrating an exemplary surgical system, according to certain embodiments.

FIGS. 3A-3C illustrate exemplary graphical user interfaces for a surgical system, according to certain embodiments.

FIG. 4 is a sequence diagram illustrating operation of an exemplary implementation of a surgical system, according to certain embodiments.

FIG. 5 is a sequence diagram illustrating operation of exemplary implementations of a surgical system, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

While the embodiments are described with respect to the specific example of an ophthalmic procedure such as phacoemulsification, the techniques described may be used in conjunction with any surgical or clinical procedure that is capable of being performed through the use of multiple surgical consoles.

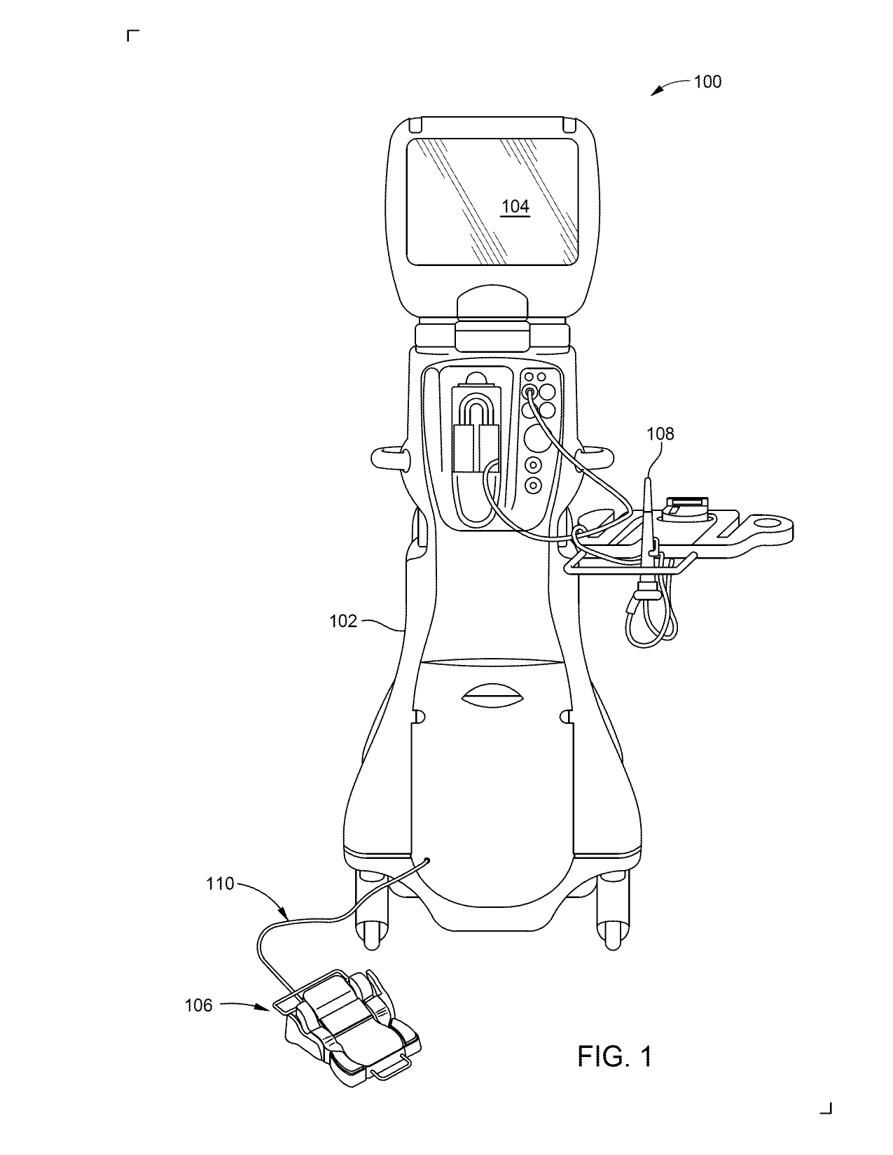

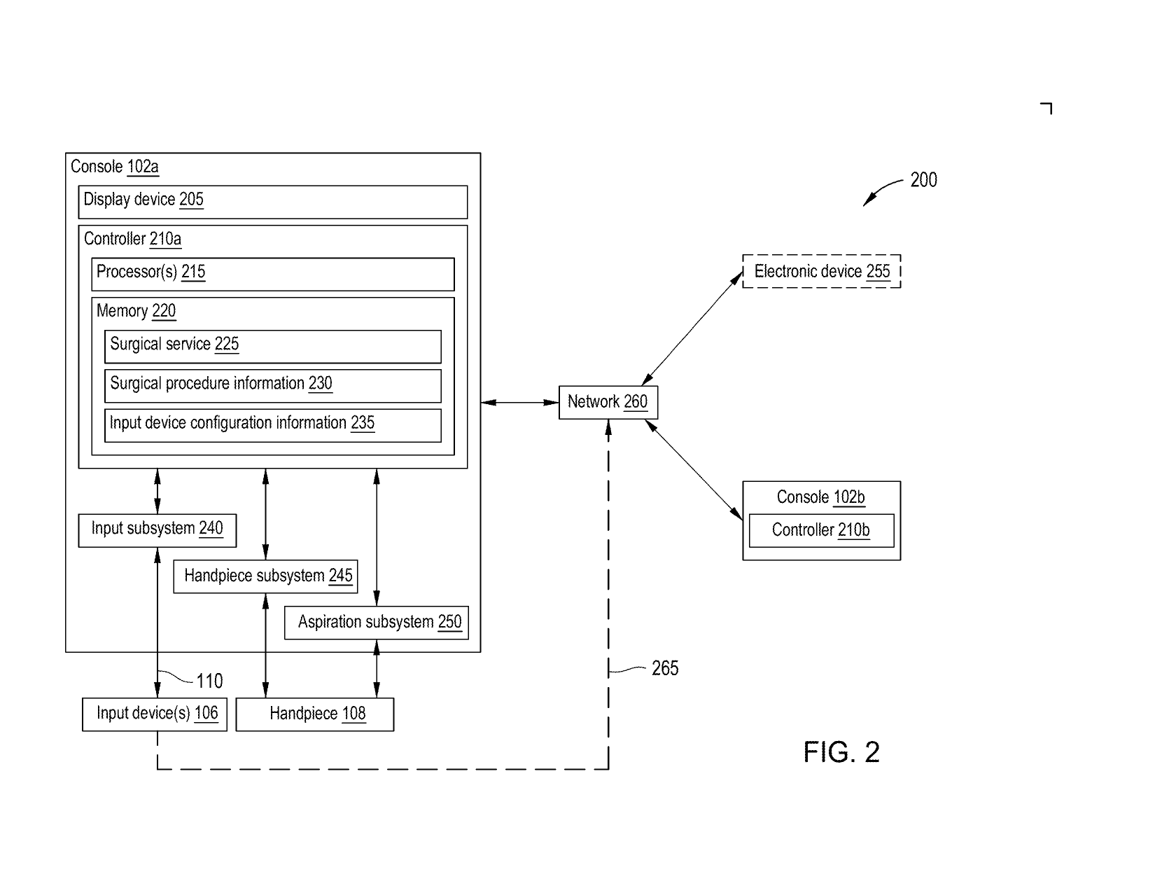

FIG. 1 illustrates an exemplary surgical console 102 (also referred to as a “console 102”) of a surgical system 100 (also referred to as a “system 100”), according to certain embodiments. More specifically, the surgical system 100 represents an example ophthalmic surgical system that may be used, for example, to perform ophthalmic procedures on an eye, according to certain embodiments. The surgical system 100 includes the console 102, which includes a display 104, an input device 106 (e.g., a foot controller), and a handpiece 108. The components of the surgical system 100 and the surgical console 102 are mechanically and/or electrically coupled as shown and described in more detail with reference to FIG. 2. Note that the depiction of console 102 is merely exemplary and that any type or brand of console or device used during a medical procedure is within the scope of the disclosure. Further, although certain embodiments herein refer to ophthalmic surgical systems and/or consoles, any console, device, or equipment that is controlled by an input device (e.g., foot controller 106, remote control, etc.) for any type of medical procedure (e.g., cardiovascular surgeries, neurosurgeries, etc.) are within the scope of the disclosure.

FIG. 2 is a block diagram illustrating an exemplary surgical system 200 (also referred to as a “system 200”), according to certain embodiments. The features of the surgical system 200 may be used in conjunction with other embodiments. For example, the surgical system 200 may include several examples of the console 102 of FIG. 1.

The system 200 comprises a first surgical console 102a and a second surgical console 102b (each representing an example of a console, such as the console 102 of FIG. 1) that are communicatively coupled with each other through a network 260. In some embodiments, the first surgical console 102a and a second surgical console 102b provide different functionality (e.g., are used for different phases of a certain surgical procedure or are used for two different surgical procedures). In one example implementation, the first surgical console 102a is a phacoemulsification console, and the second surgical console 102b is a vitreo-retinal surgical console. Other types and combinations of surgical consoles are also contemplated, which can depend on the particular surgical procedure.

The network 260 may have any suitable implementation, such as one or more wide area networks (WANs), one or more local access networks (LANs), or combinations thereof. The network 260 comprises infrastructure for communicative capability, such as conductive cabling, wireless transmission, optical transmission, and so forth. The network 260 may further comprise one or more electronic devices providing network functionality and/or services to the network 260, such as routers, firewalls, switches, gateway computers, edge servers, and so forth. In some embodiments, an electronic device 255 is communicatively coupled with the first surgical console 102a and/or the second surgical console 102b through the network 260. Although two consoles 102a, 102b are shown, alternate embodiments of the system 200 may include different numbers of consoles (e.g., three or more).

As shown, the first surgical console 102a includes a display device 205, a controller 210a, an input subsystem 240 and a handpiece subsystem 245. The display device 205 may have any suitable implementation (e.g., integrated into the console 102 as shown in FIG. 1) and use any suitable display technology. In some embodiments, the display device 205 further includes an integrated input device (e.g., implemented as a resistive or capacitive touch-screen).

The controller 210a controls the operation of the first surgical console 102a and is illustrated as being operationally coupled by a wired or wireless connection to the input device 106 via the input subsystem 240, and to the handpiece 108 via the handpiece subsystem 245. The controller 210a includes one or more processor(s) 215 and a memory 220.

The one or more processors 215 may be any type of general purpose processor or could be a processor specifically designed for driving the subsystems illustrated in FIG. 2, such as an application-specific integrated circuit (“ASIC”). The one or more processors 215 may be, or may include, a microprocessor, a microcontroller, an embedded microcontroller, a programmable digital signal processor, or any other programmable device operable to execute instructions stored in the memory 220 for operating the first surgical console 102a, or combinations thereof. For example, the one or more processors 215 may execute instructions in the memory 220 to receive inputs provided by input device 106 through the input subsystem 240 and, in response, send instructions to the various subsystems, such as the handpiece subsystem 245 for operating the handpiece 108. Further, the one or more processors 215 may execute instructions to generate a user interface view for display by the display device 205. In some instances, the one or more processors 215 may also be, or may include, a programmable gate array, programmable array logic, or any other device of combinations of devices operable to process electric signals.

The memory 220 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 220 stores instructions executed by the one or more processors 215. In example embodiments, functionality disclosed herein can be provided by the one or more processors 215 and the memory 220 (i.e., software-based), by other controller circuitry included in the controller 210a (i.e., hardware-based), or by a combination thereof. The memory 220 may include code stored thereon. The code may include instructions that may be executable by the one or more processors 215. The code may be created, for example, using any programming language, including but not limited to, C, C++, Java, Python, Rust, or any other programming language (including assembly languages, hardware description languages, and database programming languages). In some instances, the code may be a program that, when executed by the one or more processors 215, causes the first surgical console 102a to operate the different subsystems 240, 245, and/or 250 for, e.g., driving the handpiece 108 or other devices in communication with the first surgical console 102a.

The handpiece 108 may be any suitable ophthalmic surgical instrument that can be operated on the basis of the embodiments described herein. For example, the handpiece 108 may be a vitrectomy handpiece (also referred to as a “vitrectomy probe”), an irrigation handpiece, an aspiration handpiece, a laser handpiece, or combinations thereof.

The handpiece subsystem 245 is configured to facilitate the operation of the handpiece 108. For example, the handpiece subsystem 245 may control the operations (e.g., oscillatory motion) of a component (e.g., cutting assembly) of the handpiece 108.

The aspiration subsystem 250 provides aspiration control for the handpiece 108. In some embodiments, the aspiration subsystem 250 may be operatively coupled to a surgical cassette during a surgical procedure. For example, the surgical cassette may be inserted into, attached to, and/or integrated with the aspiration subsystem 250 via a coupling mechanism. When the aspiration subsystem 250 is operatively coupled to a surgical cassette, the aspiration subsystem 250 may control aspiration through the surgical cassette, which may in turn be coupled to the handpiece 108, for example, via a cable or other tether. In certain embodiments, the aspiration subsystem 250 includes one or more mechanical pumps having roller pump heads configured to engage with one or more corresponding pump assemblies on the surgical cassette. The engagement of the roller pump heads and pump assemblies generates a source of pressure and/or vacuum utilized during an ophthalmic surgical procedure.

The input device 106 (also referred to as an “electronic accessory”) may be any device that is capable of receiving commands from the user of the first surgical console 102a in order to operate the handpiece 108 and/or provide control to other components of the first surgical console 102a. In FIG. 1, the input device 106 is illustrated as a foot controller, however, other types of input devices are also within the scope of the disclosure. In one example, the user provides a command to the input device 106 (e.g., a surgeon using his or her foot to manipulate a pedal of the foot controller), which is received by the input subsystem 240 and relayed to the controller 210a. In response, the controller 210a sends instructions to the handpiece subsystem 245 and/or aspiration subsystem 250 to control the operations of the handpiece 108 based on the user command.

The memory 220 comprises a surgical service 225 representing code that is executed by the one or more processors 215 to cause the first surgical console 102a to operate the different subsystems 240, 245, and/or 250 for receiving inputs from the input device 106, driving the handpiece 108, and so forth. The surgical service 225 may further cause the one or more processors 215 to display one or more graphical elements to the display device 205 (e.g., displaying a user interface) to provide information to the surgeon, to prompt the surgeon for information, and so forth.

In some embodiments, the memory 220 stores surgical procedure information 230 describing, for one or more surgical procedures, a predefined sequence of phases, steps, etc. to be performed by the surgeon for the surgical procedure. In some embodiments, the surgical service 225 monitors inputs received at the input device 106 and/or the user interface to determine a current phase or step of the surgical procedure. In some embodiments, the surgical service 225 controls the appearance of the user interface based on the surgical procedure information 230 and/or the received inputs (e.g., displaying the current phase or step on the display device 205, displaying different input options based on the current phase or step). In some embodiments, the surgical service 225 may control the functionality of the input device 106 based on the surgical procedure information 230 and/or the received inputs. For example, an input received by the controller 210a during a first phase of the surgical procedure may be interpreted to cause a first action (e.g., at the handpiece 108), and the same input received during a second phase of the surgical procedure may be interpreted to cause a second action.

Conventionally, the input device 106 can be considered as being “owned” or “operationally assigned” to the surgical console to which it is connected (here, the first surgical console 102a). In order to use the input device 106 to operate another surgical console (say, the second surgical console 102b), the input device 106 is conventionally disconnected from the first surgical console 102a (unplugged and/or disconnected via the user interface), connected with the second surgical console 102b (plugged and/or connected via the user interface), and in some cases reconfigured at the second surgical console 102b. Such a process can be time-consuming and susceptible to errors, making it inefficient and likely unsuitable for the surgical environment.

In some embodiments, the surgical service 225 manages the “ownership” or operational assignment of the input device 106. The surgical service 225 may initiate or perform a “handoff” of the input device 106, such that the input device 106 may be assigned to operate different surgical consoles at different times. In this way, the same input device 106 may be more easily transitioned between the first surgical console 102a and the second surgical console 102b without requiring physical disconnection and/or a complex reconfiguration of the input device 106. Using the surgical service 225 to manage ownership of the input device 106 saves time, reduces the risk of errors, helps the surgeon maintain focus on the procedure, and so forth.

In some embodiments, during a first time period in which the first surgical console 102a is assigned ownership of the input device 106 (e.g., the controller 210a claims ownership or control of the input device 106), the controller 210a (e.g., the surgical service 225) controls operations of the first surgical console 102a based on inputs received from the input device 106. For example, the first time period may include a time period during which a particular operation (vitreo-retinal surgery) or phase of an operation is performed on a patient and/or completed. During the first time period, the second surgical console 102b is non-responsive to the inputs received from the input device 106. The second surgical console 102b being “non-responsive” to the inputs encompasses a case where the inputs are not received by the second surgical console 102b, as well as a case where the inputs are received by the second surgical console 102b but are disregarded. For example, the second surgical console 102b may determine to be non-responsive to the inputs when not assigned ownership of the input device 106.

The controller 210a receives (e.g., during the first time period) a transfer indication that indicates that ownership of the input device 106 should be transferred to the second surgical console 102b. In some embodiments, the transfer indication is received through a user interface of the first surgical console 102a, e.g., as an instruction to handoff the ownership of the input device 106. In some embodiments, the transfer indication is received through a user interface of the second surgical console 102b, e.g., as a request to handoff the ownership of the input device 106. In some embodiments, the transfer indication is received through a user interface of the electronic device 255. In some embodiments, the transfer indication is received as a signal that is generated responsive to completion of a phase of a surgical procedure.

As shown in FIG. 1, the input device 106 is connected with the first surgical console 102a through a wired connection 110, which may encompass one or more conductors (e.g., wires), one or more optical fibers, and so forth. In some embodiments, the wired connection 110 carries one or more signals between the input device 106 and the first surgical console 102a (e.g., a configuration signal transmitted by the first surgical console 102a, a command signal transmitted by the input device 106 responsive to the input received at the input device 106). In some embodiments, the wired connection 110 (also) carries electrical power from the first surgical console 102a to the input device 106.

In some embodiments, the input device 106 is connected with the first surgical console 102a through a wireless connection 265, which may be in addition to the wired connection 110 or alternate to the wired connection 110. The wireless connection 265 may encompass radio frequency signals, microwave signals, free-space optical signals, and so forth. In some embodiments, the input device 106 is communicatively coupled to the network 260 via the wireless connection 265.

The optional electronic device 255 may have any suitable implementation. As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. In some embodiments, the electronic device 255 is implemented as a mobile computing device such as a handheld device (e.g., smartphone or tablet) or laptop computer that displays a user interface and/or receives inputs. The electronic device 255 communicates with the first surgical console 102a and/or the second surgical console 102b. In this way, the electronic device 255 may be used by the surgeon or technicians in addition to, or alternate to, the display device 205 and any associated input device.

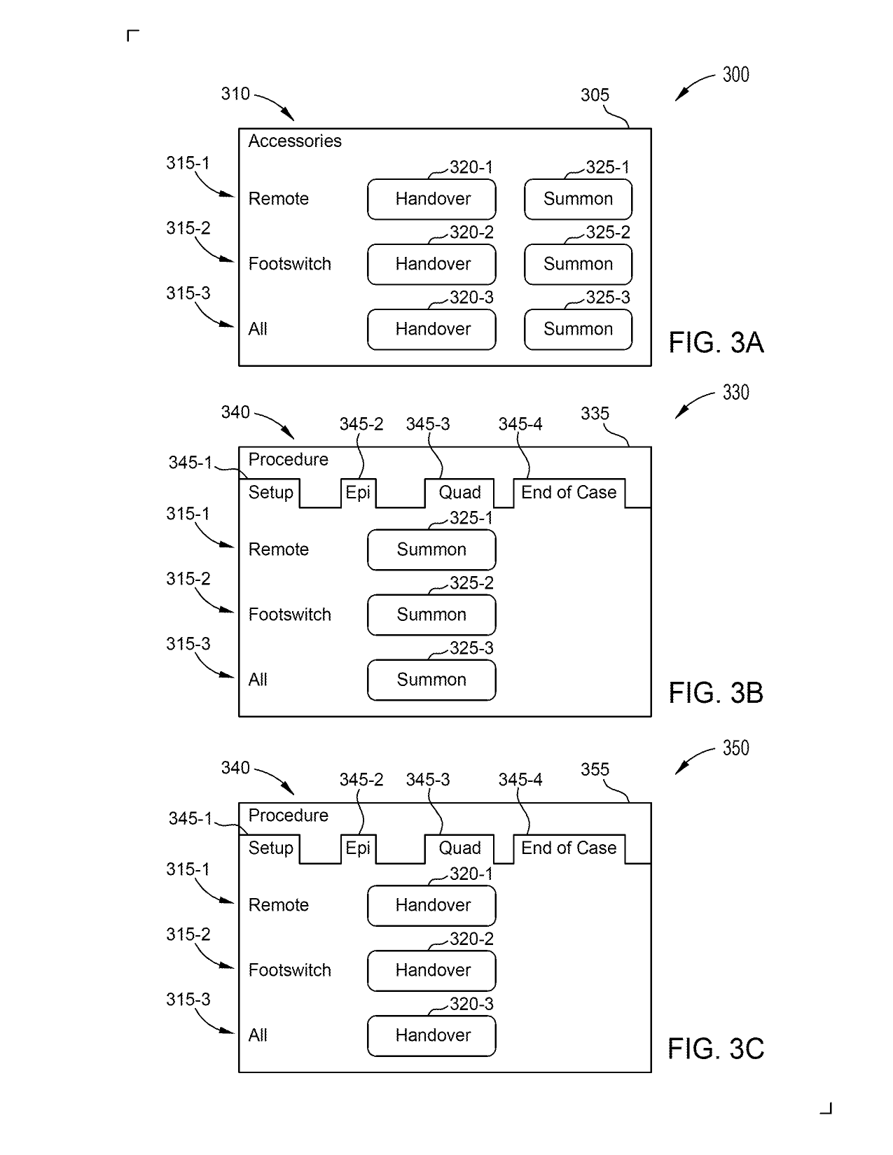

Examples of user interfaces that may be used in conjunction with one or more of the handoff procedures described herein are illustrated in FIGS. 3A-3C. For example, the user interfaces in FIGS. 3A-3C may be generated on one or more devices/consoles of the surgical system 200, such as by the surgical service 225 and displayed by the display device 205 of the first surgical console 102a, a display device of the second surgical console 102b, or a display device of the electronic device 255. In some embodiments, the user interfaces may be used to generate the transfer indication within the surgical system 200. For example, the transfer indication may be caused by a finger press or other selection of a button displayed on the user interface.

The user interface (UI) 300 of FIG. 3A displays a boundary 305 and a title 310 (“Accessories”). Within the boundary 305, the UI 300 displays a first label 315-1 corresponding to a first input device (“Remote”) that may be operated with the first surgical console 102a and with the second surgical console 102b, and a second label 315-2 corresponding to a second input device (“Footswitch”) that may be operated with the first surgical console 102a and with the second surgical console 102b. In some embodiments, the UI 300 further displays a third label 315-3 corresponding to both of the input devices (“All”).

The UI 300 further displays a first set of buttons 320-1, 320-2, 320-3 (“Handover”), corresponding to the labels 315-1, 315-2, 315-3, which when selected by a user, instruct the surgical service 225 to handoff the ownership of the respective input device(s). The UI 300 further displays a second set of buttons 325-1, 325-2, 325-3 (“Summon”), corresponding to the labels 315-1, 315-2, 315-3, which when selected by a user, request the surgical service 225 to handoff the ownership of the respective input device(s).

In some embodiments, the UI 300 represents a default UI (e.g., non-specific to a surgical procedure) that the surgical service 225 causes to be displayed. In some embodiments, the UI 300 is always displayed by the surgical system 200. In some embodiments, the UI 300 is displayed by the surgical system 200 when none of the surgical procedures are being actively performed. In some embodiments, the UI 300 is displayed by the surgical system 200 prior to commencement and/or upon completion of a surgical procedure.

The UI 330 of FIG. 3B displays a boundary 335 and a title 340 (“Procedure”). In some embodiments, the name of the surgical procedure may be displayed as the title 340 (e.g., “Phaco” for phacoemulsification) or elsewhere in the UI 330. Within the boundary 335, the UI 330 further displays a plurality of tabs 345-1 (“Setup”), 345-2 (“Epi” for epinucleus removal), 345-3 (“Quad” for quadrant removal), 345-4 (“End of Case”) representing various phases or steps of the surgical procedure. In some embodiments, the content displayed in the plurality of tabs 345-1, 345-2, 345-3, 345-4 may be determined by accessing the surgical procedure information 230. In some embodiments, the tabs 345-2, 345-3 represent procedural phases or steps defined in the surgical procedure retrieved from the surgical procedure information 230, and the tabs 345-1, 345-4 represent administrative phases or steps that are appended to the beginning and end of the procedural phases or steps.

Although not shown in FIG. 3B, individual ones of the plurality of tabs 345-1, 345-2, 345-3, 345-4 may be visually highlighted (e.g., a bold outline, bold text, other tabs de-emphasized) to show which phase or step is active. In some embodiments, the phase or step may be selectable through user input received at the respective tabs 345-1, 345-2, 345-3, 345-4.

The UI 330 of FIG. 3B includes the second set of buttons 325-1, 325-2, 325-3 (“Summon”) and omits the first set of buttons 320-1, 320-2, 320-3. In alternate embodiments, the first set of buttons 320-1, 320-2, 320-3 may be displayed (as in the UI 300) but de-emphasized (e.g., grayed out) or otherwise disabled from receiving inputs.

In some embodiments, the surgical service 225 causes the UI 330 to be displayed on a particular surgical console when the other surgical console has ownership of the input device(s). In one non-limiting example, the console 102a is configured to perform irrigation and vitrectomy functions within a surgical procedure, and the console 102b is configured to perform a later aspiration function. The console 102b may display the “Summon” button(s) while the console 102a “owns” the input device 106. In some embodiments, the surgical service 225 causes the UI 330 to be displayed based on the active phase or step, which in some cases can be independent of the current ownership assignment of the input device(s). Adapting the previous example, the console 102a is configured to perform irrigation and vitrectomy functions within a surgical procedure, and the console 102b is configured to perform a later aspiration function. The console 102b may display the “Summon” button(s) while the irrigation or vitrectomy functions are active.

The user interface (UI) 350 of FIG. 3C displays a boundary 355, the title 340 (“Procedure”), the plurality of tabs 345-1, …, 345-4. The UI 350 of FIG. 3C includes the first set of buttons 320-1, 320-2, 320-3 and omits the second set of buttons 325-1, 325-2, 325-3. In alternate embodiments, the second set of buttons 325-1, 325-2, 325-3 may be displayed (as in the UI 300) but de-emphasized (e.g., grayed out) or otherwise disabled from receiving inputs.

In some embodiments, the surgical service 225 causes the UI 350 to be displayed on a particular surgical console when the particular surgical console has ownership of the input device(s) and/or the other surgical console does not have ownership of the input device(s). In some embodiments, the surgical service 225 causes the UI 350 to be displayed based on the active phase or step of the surgical procedure, which in some cases can be independent of the current ownership assignment of the input device(s). Adapting the previous example, the console 102a may display the “Handover” button(s) while the irrigation or vitrectomy functions are active.

In the example UIs 300, 330, 350, the first set of buttons 320-1, 320-2, 320-3 and/or the second set of buttons 325-1, 325-2, 325-3 are displayed in their entirety. However, other displays of the various buttons are also contemplated. For example, a “handover” button 320-1 and a “summon” button 325-2 may be displayed by the first surgical console 120-1 when the remote is owned by the first surgical console 120-1 but the foot controller is owned by the second surgical console 120-2. The “handover” button 320-3 and the “summon” button 325-3 (corresponding to the “All” label 315-3) may or may not be displayed in this example.

In some embodiments, the surgical service 225 may assign ownership of the different input device(s) based on the active phase or step of the surgical procedure. For example, when the “End of Case” phase (tab 345-4) becomes active, indicating the completion of the surgical procedure, the ownership of the input device(s) may revert to the surgical console having original ownership, to a surgical console that is assigned priority, and so forth.

Returning to FIG. 2, in some embodiments, responsive to the controller 210a receiving the transfer indication, in some embodiments, the surgical service 225 transmits a prompt (e.g., to the display device 205) seeking confirmation of the transfer. In such embodiments, responsive to receiving the confirmation (e.g., a user input), the surgical service 225 initiates a transfer of the ownership of the input device 106 to the second surgical console 102b. In some other embodiments, the transfer of ownership may be performed without transmitting the prompt and receiving a confirmation.

The transfer of ownership may be performed in any suitable manner, which may depend on the type of connection between the input device 106 and the first surgical console 102a, and/or the communication protocols employed by the network 260.

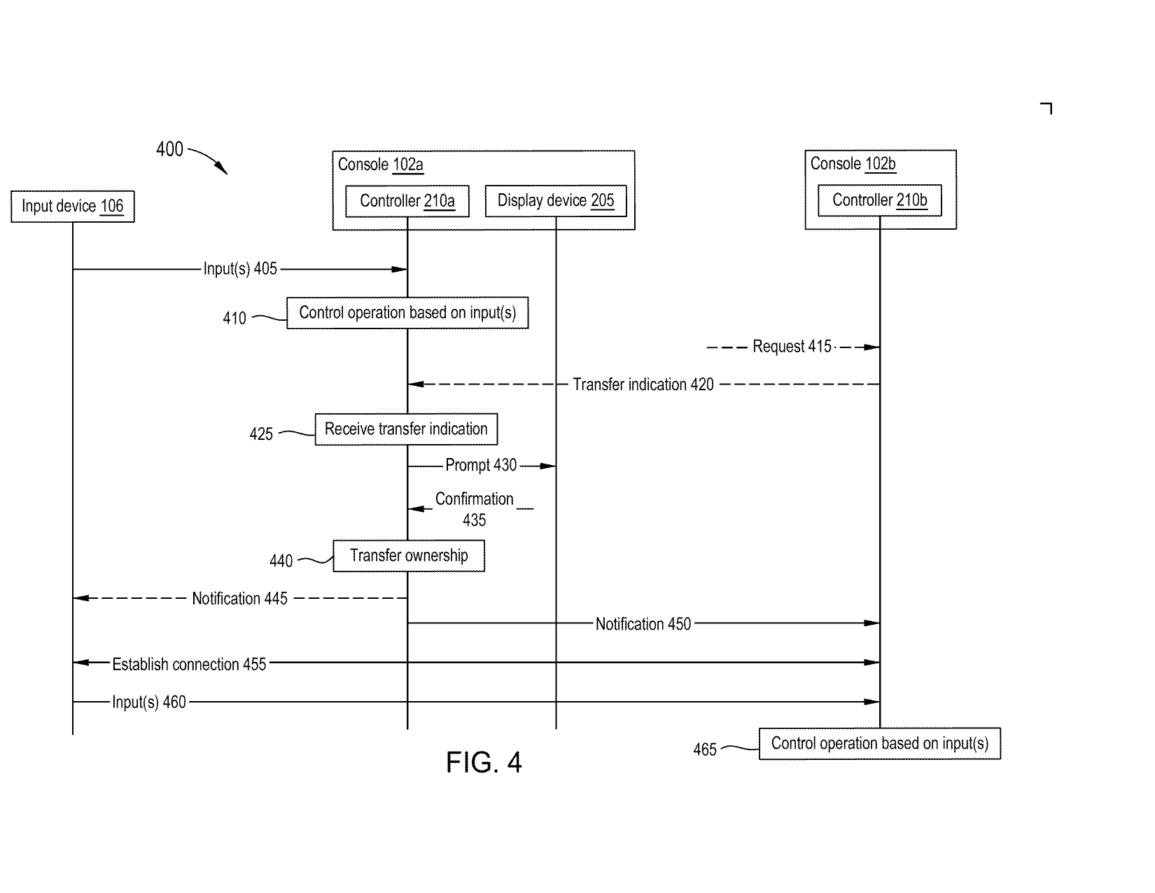

FIG. 4 is a sequence diagram 400 illustrating operation of one or more exemplary implementations of a surgical system, according to certain embodiments. The features of the sequence diagram 400 may be used in conjunction with other embodiments, for example, performed using various actors included in the surgical system 200.

In one example implementation, the input device 106 is wirelessly coupled (through the wireless connection 265) to the first surgical console 102a. The first surgical console 102a and the second surgical console 102b are wirelessly coupled to the network 260. In some embodiments, the input device 106 is wirelessly coupled to the first surgical console 102a through the network 260. In other embodiments, the wireless connection 265 is separate from the network 260 (e.g., using different frequencies, channels, protocols, etc.).

In some embodiments, the wireless connection 265 uses protocols in which the input device 106 is paired (and/or bonded) with one of the first surgical console 102a and the second surgical console 102b at any given time. The protocols may be standardized (e.g., Bluetooth, Bluetooth Low Energy (BLE), other short-range communication protocols, etc.) or proprietary.

During a first time period, the first surgical console 102a is assigned ownership of the input device 106 by the controller 210a (e.g., the surgical service 225). As such, when one or more inputs 405 are transmitted by the input device 106 and received by the controller 210a during the first time period, at block 410, the controller 210a controls operation of the first surgical console 102a based on the input(s) 405. In some embodiments, the first time period represents the duration of time that the first surgical console 102a is assigned ownership of the input device 106.

At block 425, the controller 210a receives or generates a transfer indication. In some embodiments, a request 415 is optionally received at the second surgical console 102b (e.g., a press of a “Summon” button at the UI of the second surgical console 102b), and the controller 210a of the second surgical console 102b transmits the transfer indication 420 to the controller 210a. In some embodiments, the transfer indication is received by the controller 210a from a user interface of console 102a (e.g., a press of a “Handover” button at the UI of the first surgical console 102a) or received from an external electronic device. In some other embodiments, the controller 210a itself generates the transfer indication, which can be as simple as setting or clearing a bit in the memory 220. For example, the controller 210a may generate the transfer indication responsive to determining that a phase or step of a surgical procedure has been completed (e.g., an input received at the UI indicating the end of the phase or step).

In some embodiments, the controller 210a transmits a prompt 430 to the display device 205 seeking a confirmation of the transfer. In some other embodiments, the prompt 430 may be transmitted to and then displayed by a requesting device/console (e.g., at the second surgical console 102b or an external electronic device).

Responsive to receiving a confirmation 435, at block 440, the controller 210a transfers ownership of the input device 106 to the second surgical console 102b. In some embodiments, transferring ownership of the input device 106 may correspond to the end of the first time period.

In some embodiments, transferring ownership includes unpairing and/or disconnecting from the input device 106. In some embodiments, transferring ownership includes determining to be unresponsive to any inputs received from the input device 106. In some embodiments, the controller 210a may continue tracking the ownership of the input device 106 (e.g., storing information in the memory 220) across the different surgical consoles 102a, 102b. In other embodiments, the controller 210a does not track the ownership (e.g., where the controller 210a unpairs and/or disconnects from the input device 106).

In some embodiments, transferring ownership includes sending notifications 445 and/or 450 to input device 106 and/or the second surgical console 102b, respectively. For example, in some embodiments, the controller 210a transmits the notification 445 to the input device 106, and/or transmits the notification 450 to the second surgical console 102b about the ownership transfer. In some embodiments, the notifications 445, 450 indicate that the first surgical console 102a is unpairing from the input device 106. In some embodiments, the notifications 445, 450 may include information that assists the input device 106 and/or the controller 210b with establishing a connection between the input device 106 and the second surgical console 102b, e.g., identifying information associated with input device 106 and/or the second surgical console 102b (e.g., a device name or unique identifier of the second surgical console 102b, a network address that may be software or hardware-based (e.g., an IP (Internet Protocol) address or MAC (Media Access Control) address) that the input device 106 can use to establish a subsequent connection with the second surgical console 102b). In some embodiments, the notifications 445, 450 may be visually displayed using a display device of the second surgical console 102b, an external electronic device, etc.

Subsequently, the input device 106 and the controller 210b establish a connection 455 with each other. In some embodiments, the input device 106 and the second surgical console 102a use information received in the notifications 445, 450, e.g., the input device 106 receives a network address from the first surgical console 102a that pre-configures the input device 106 to pair with the second surgical console 102b. In some embodiments, establishing the connection 455 includes pairing and/or bonding of the input device 106 with the second surgical console 102b (e.g., with the controller 210b) according to, for example, certain short-ranged communication protocols (e.g., Bluetooth/BLE).

During a second time period in which the second surgical console 102b is assigned ownership of the input device 106 (e.g., the controller 210b claims ownership or control of the input device 106), the controller 210b receives one or more inputs 460 from the input device 106. In some embodiments, the second time period represents the duration of time that the second surgical console 102b is assigned ownership of the input device 106. In certain embodiments, the first time period and the second time period may overlap and, in certain other embodiments, they may not.

At block 465, the controller 210b controls operation of the second surgical console 102b based on the inputs 460. During the second time period, the first surgical console 102a is non-responsive to the one or more inputs 460. For example, in embodiments where the first surgical console 102a has previously terminated its communication with the input device 106, the first surgical console 102a does not receive the one or more inputs 460 and/or disregards them. In some embodiments, another transfer of ownership of the input device 106 may correspond to the end of the second time period.

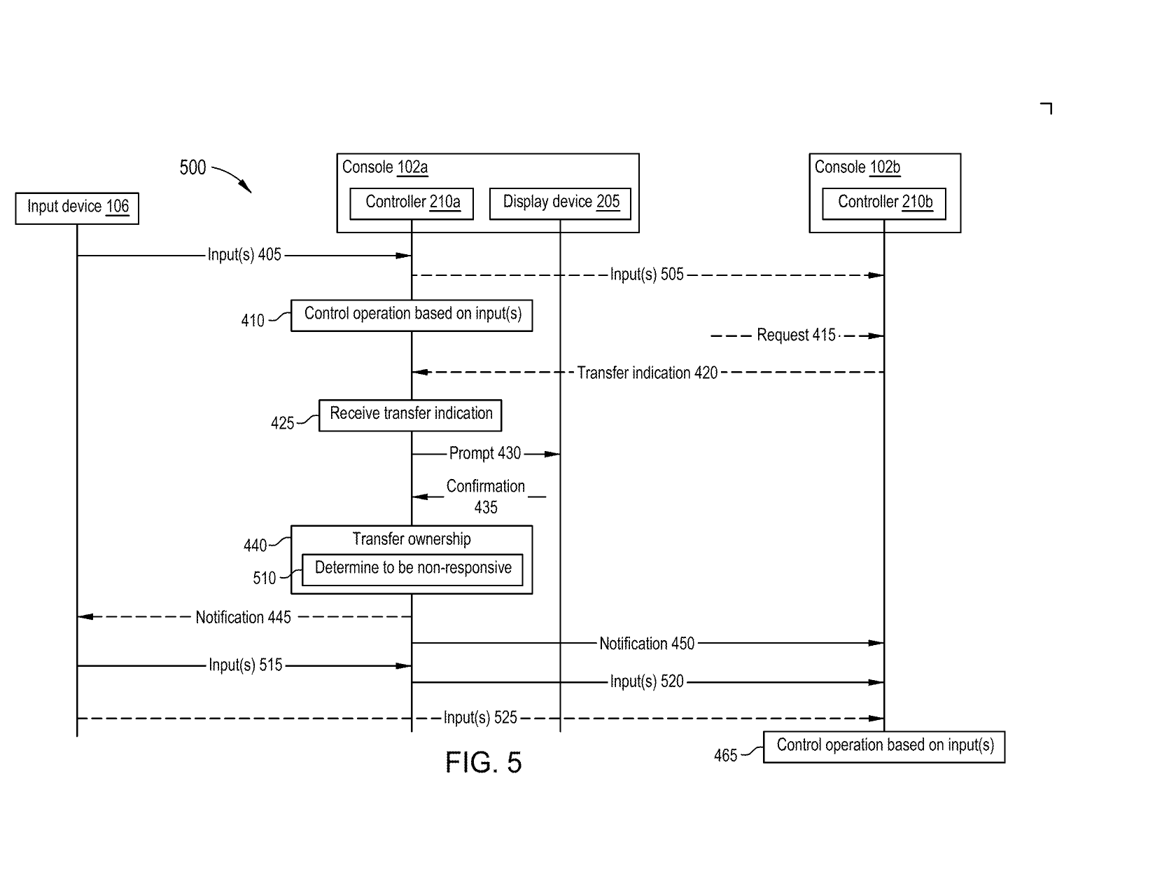

FIG. 5 is a sequence diagram 500 illustrating operation of one or more exemplary implementations of a surgical system, according to certain embodiments. The features of the sequence diagram 400 may be used in conjunction with other embodiments, for example, performed using various actors included in the surgical system 200.

In one example implementation, the input device 106, the first surgical console 102a, and the second surgical console 102b are wirelessly coupled to the network 260. In some embodiments, the network 260 uses protocols in which communications may be broadcast by one or more of the network-connected devices. The protocols may be standardized (e.g., IEEE (Institute of Electrical and Electronics Engineers) 802.15.4) or proprietary. In some embodiments, the first surgical console 102a may be assigned a coordinating or supervisory role according to the protocols, whether the network 260 has a peer-to-peer topology (e.g., a network coordinator) or a star topology (e.g., a hub). In some embodiments, the first surgical console 102a may be assigned a higher-priority role than other surgical consoles (e.g., the second surgical console 102b) of the surgical system 200. For example, the first surgical console 102a when assigned a higher-priority role may be configured to broadcast status information onto the network 260 periodically to confirm safe and reliable operation of the surgical system 200.

In another example implementation consistent with the sequence diagram 500, the input device 106 has a wired connection 110 to the first surgical console 102a, and the first surgical console 102a and the second surgical console 102b are wirelessly coupled with each other through the network 260. Thus, in some embodiments, the controller 210a by virtue of its wired connection 110, its coordinating or supervisory role, and/or its higher-priority role, operates as a broker (or intermediary) that translates the input(s) 405, 515 between two different connection types, two different communications protocols, etc.

In another example implementation consistent with the sequence diagram 500, the input device 106 and the first surgical console 102a are wirelessly coupled with each other using a first set of wireless protocol(s), and the first surgical console 102a and the second surgical console 102b are wirelessly coupled with each other through a second set of wireless protocol(s). In this implementation, the second surgical console 102b may not be configured to use the first set of wireless protocol(s). In one non-limiting example, the first set of wireless protocol(s) may include Bluetooth or BLE (e.g., requiring relatively less power consumption) and the second set of the wireless protocol(s) may include IEEE 802.15.4 or other protocol(s) that are different from the first set. Thus, the controller 210a may perform a “translation” function between the first set and the second set, adapting and/or repackaging the information received from the input device 106 into a form suitable for the controller 210b.

During the first time period in which the first surgical console 102a is assigned ownership of the input device 106, the one or more inputs 405 are transmitted by the input device 106 and received by the controller 210a. In some embodiments, the controller 210b of the second surgical console 102b may also receive the one or more inputs 405 but be configured to disregard them as the second surgical console 102b does not have ownership of the input device 106 during the first time period. In some embodiments, the controller 210a may be configured to receive the one or more inputs 405 and retransmit (broadcast) them as one or more inputs 505. In certain embodiments, the one or more inputs 505 may be modified or unaltered versions of the one or more input 505.

At block 410, the controller 210a controls operation of the first surgical console 102a based on the input(s) 405. During the first time period, the second surgical console 102b is non-responsive to the one or more inputs 405. For example, while the second surgical console 102b may receive the input(s) 405, the second surgical console 102b can be configured to be non-responsive to the input(s) 405 (e.g., to disregard the input(s) 405). In other embodiments, the modification(s) made by the controller 210a to form the input(s) 505 may indicate to the second surgical console 102b that the first surgical console 102a is assigned ownership of the input device 106, or that may configure the second surgical console 102b to be non-responsive to the input(s) 505.

The sequence diagram 500 continues in a similar manner to the sequence diagram 400 until block 440, where the controller 210a transfers ownership of the input device 106 to the second surgical console 102b. In some embodiments, transferring ownership of the input device 106 comprises, at block 510, the first surgical console 102a (e.g., the controller 210a) determining to be non-responsive to the input(s) 405 (e.g., to disregard the input(s) 405). In some embodiments, transferring ownership includes sending notifications 445 and/or 450 to the input device 106 and/or the second surgical console 102b, respectively.

After transmitting the notifications 445, 450, the input device 106 and the second surgical console 102b may omit establishing the connection 455 as the second surgical console 102b is already able to receive information broadcasted by input device 106 and/or retransmitted/rebroadcasted by the controller 210a.

During the second time period in which the second surgical console 102b is assigned ownership of the input device 106, the controller 210a receives one or more inputs 515 from the input device 106. In some embodiments, the controller 210a retransmits the information from the one or more inputs 515 (e.g., broadcasts) to the controller 210b as one or more inputs 520. In some embodiments, the controller 210a may modify the input(s) 515 when transmitting the input(s) 520. In other embodiments, the input device 106 may itself broadcast the one or more inputs 515, 525 to the controllers 210a, 210b, respectively, such that retransmission by the controller 210a would be redundant.

At block 465, the controller 210b controls operation of the second surgical console 102b based on the input(s) 515, 520, 525. In this implementation, the first surgical console 102a is non-responsive to the input(s) 515. While the first surgical console 102a may receive the information from the input(s) 515, the first surgical console 102a has determined at block 510 to be non-responsive to the input(s) 405 (e.g., to disregard the input(s) 405).

In some embodiments, the first surgical console 102a may be responsible for communication with the input device 106 regardless of whether the input device 106 is being used to control the first surgical console 102a or second surgical console 102b. For example, the first surgical console 102a may receive input(s) and be controlled by input device 106 when input device 106 is assigned to the first surgical console 102a. Then, upon the input device 106 being transferred to the second surgical console 102b, the input device 106 may continue to communicate with and be controlled by the first surgical console 102a which may act to pass through the input(s) received from the input device 106 to the second surgical console 102(b) and pass through communications from the second surgical console 102(b) to the input device 106. In this embodiment, the first surgical console 102a may also, for example, receive a configuration from the second surgical console 102b to be used by the input device 106, and the first surgical console 102a may pass the configuration through to (or implement the configuration itself) the input device 106 to be used in communicating with the second surgical console 102b. In some embodiments, the first surgical console 102a and the second surgical console 102b may both receive inputs from the input device 106 directly (or through a controller as discussed above), but communication from the first surgical console 102a and/or the second surgical console 102b to the input device 106 may be through only one of the surgical consoles (for example, only the first surgical console 102a may communicate to the input device 106 even when the input device 106 is assigned to the second surgical console 102b). Other configurations are also contemplated.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A surgical system comprising:
an input device; and
a first surgical console and a second surgical console communicatively coupled with each other,
wherein the first surgical console comprises a first controller configured to:
receive a first input from the input device during a first time period in which the first surgical console is assigned ownership of the input device, wherein the second surgical console is non-responsive to the first input during the first time period;
control operations of the first surgical console based on the first input; and
transfer, responsive to receiving a transfer indication, the ownership of the input device to the second surgical console;
wherein the second surgical console comprises a second controller configured to:
receive a second input from the input device during a second time period in which the second surgical console is assigned ownership of the input device, wherein the first surgical console is non-responsive to the second input during the second time period;
control operations of the second surgical console based on the second input.

2. The surgical system of claim 1, wherein the transfer indication is received through a user interface of the first surgical console.

3. The surgical system of claim 2, wherein the transfer indication is received responsive to completion of a phase of a surgical procedure.

4. The surgical system of claim 1, wherein the transfer indication is received from an electronic device responsive to a request provided through a user interface of the electronic device.

5. The surgical system of claim 1, wherein the transfer indication is received from the second surgical console responsive to a request provided through a user interface of an electronic device.

6. The surgical system of claim 1, wherein transferring the ownership of the input device to the second surgical console comprises:
notifying the second surgical console of the transfer of ownership.

7. The surgical system of claim 1, wherein transferring the ownership of the input device to the second surgical console comprises:
notifying the input device of the transfer of ownership.

8. The surgical system of claim 1, wherein transferring the ownership of the input device to the second surgical console comprises:
determining to be non-responsive to inputs received from the input device subsequent to the transfer.

9. A surgical console comprising:
one or more processors; and
a memory storing instructions that when executed by the one or more processors enable performance of an operation comprising:
receiving inputs from an input device during a first time period in which the surgical console is assigned ownership of the input device;
controlling operations of the surgical console based on the inputs; and
wherein the surgical console is a first surgical console, and wherein the performance of operation further comprises transferring, responsive to receiving a transfer indication, the ownership of the input device to a second surgical console,
wherein the second surgical console is non-responsive to the inputs received during the first time period,
wherein operations of the second surgical console are controlled based on inputs from the input device during a second time period in which the second surgical console is assigned ownership of the input device, and
wherein the first surgical console is non-responsive to the inputs received during the second time period.

10. The surgical console of claim 9, wherein the transfer indication is received through a user interface of the first surgical console.

11. The surgical console of claim 10, wherein the transfer indication is received responsive to completion of a phase of a surgical procedure.

12. The surgical console of claim 9, wherein the transfer indication is received from an electronic device responsive to a request provided through a user interface of the electronic device.

13. The surgical console of claim 9, wherein the transfer indication is received from the second surgical console responsive to a request provided through a user interface of an electronic device.

14. The surgical console of claim 1, wherein transferring the ownership of the input device to the second surgical console comprises:
notifying the second surgical console of the transfer of ownership.

15. The surgical console of claim 1, wherein transferring the ownership of the input device to the second surgical console comprises:
notifying the input device of the transfer of ownership.
